(19) United States
(12) Patent Application Publication
(10) Pub. No.: US 2010/0119499 A1
(43) Pub. Date: May 13, 2010

(54) STILBENE-BASED COMPOSITIONS AND METHODS OF USE THEREFOR

(76) Inventors: Bruce W. Kneller, Jackson, NJ (US); Daniel C. Pierce, Brooklyn, NY (US); Joseph Babick, JR., Forked River, NJ (US)

Correspondence Address: MORSE, BARNES-BROWN & PENDLETON, P.C.;ATTN: IP MANAGER, RESERVOIR PLACE, 1601 TRAPELO ROAD, SUITE 205, WALTHAM, MA, 02451, US

(21) Appl. No.: 12/687,962

(22) Filed: Jan. 15, 2010

Related U.S. Application Data
(63) Continuation of application No. PCT/US2009/057310, filed on Sep. 17, 2009.

(51) Int. Cl. A61K 38/43; A61K 31/075; A61K 31/195; A61K 38/16

(52) U.S. Cl. 424/94.1; 514/720; 514/563; 514/12

(57) ABSTRACT

Disclosed are compositions, formulations and methods relating to one or more stilbene-based compounds for use in humans. In particular, compositions and formulations comprising an effective amount of the stilbene-based insulinogenic compound can improve athletic performance, lower blood glucose levels, and increase lean muscle mass when administered (e.g., orally) to a human.

### RELATED APPLICATIONS

This application is a continuation of International Application No. PCT/US2009/57310, filed Sep. 17, 2009. The entire teachings of this application are incorporated herein by reference.

### BACKGROUND OF THE INVENTION

It is well known by those skilled in the art that the addition of various nutrients and dietary supplements to the diet of humans can greatly increase athletic performance and reduce post-recovery/post-exercise time back to baseline state. The oral use of various types of nutrients, including carbohydrates, proteins, essential amino acids and creatine for increasing athletic performance, increasing protein synthesis, enhancing tissue repair, and reducing recovery times in humans is well known (JISSN 5(17):1-12 (2008); JISSN 3(1):7-27 (2006). The importance of the timing of nutrient administration (e.g., before, during and after exercise) in both endurance exercise and resistance training has also been acknowledged. Additional dietary supplements that can increase the rate or quantity of absorption, rate or quantity of nutrient partitioning, or rate or quantity of use of various athletic performance-enhancing nutrients (including, but not limited to, carbohydrates, proteins, vitamins, minerals, amino acids, creatine by skeletal muscle cells and tissues) are desirable for improving weight loss, increasing lean muscle mass, and/or improving athletic performance.

Stilbenes are small (molecular weight of 210-278 g/mol), naturally-occurring compounds found in a wide range of plant sources, aromatherapy products, and dietary compositions. Stilbenes exist as stereoisomers in E and Z forms, depending on where functional groups are attached in relation to one another on either side of the double bond. Naturally-occurring stilbenes overwhelmingly exist in the Z (trans) form. The E and Z forms of stilbenes have different pharmacological activities and elicit different effects. Research has revealed the Z form to exhibit more potent activity compared to the E form across various anti-cancer and anti-oxidant assays. One such study demonstrated the stilbene trans-reseveratrol to be ten times more potent in its ability to induce apoptosis in the MAO leukemia cell line compared to cis-resveratrol (J Med Chem 46:3546-54 (2003)).

Other stilbenoid compounds include, but are not limited to, piceatannol, pinosylvin, rhapontigenin, tamoxifen, and pterostilbene. Pterostilbene is thought to be a key compound found predominantly in blueberries (as well as grapes) that exhibits anti-fungal, anti-cancer, anti-hypercholesterolemia, and anti-hypertriglyceridemia properties, as well as the ability to delay and reverse cognitive decline. Thus, it may be desirable to include stilbene-based compounds in dietary supplements to impart a variety of benefits.

### SUMMARY OF THE INVENTION

Disclosed herein are compositions, formulations and methods relating to one or more stilbene-based compounds for use in humans. In particular, compositions and formulations comprising an effective amount of the stilbene-based insulinogenic compound can improve athletic performance, lower blood glucose levels, and increase lean muscle mass when administered (e.g., orally) to a human. In a particular embodiment the stilbene-based compound is pterostilbene.

Thus, in one embodiment the invention relates to a method of increasing athletic performance, lowering blood sugar level and/or increasing lean muscle mass in a human comprising administering an effective amount of a stilbene-based compound to said human. In one embodiment the stilbene-based compound is pterostilbene. In one embodiment the pterostilbene is administered orally.

In certain embodiments the effective amount of pterostilbene is from about 3 mg to about 25 mg per dose, from about 3 mg to about 10 mg per dose, or from about 5 mg to about 10 mg per dose.

In certain embodiments the effective amount of pterostilbene is from about 0.03 mg/kg bodyweight of the human to about 0.165 mg/kg bodyweight of the human per dose.

In certain embodiments more than one dose comprising an effective amount of pterostilbene is administered to the human (e.g., two or more doses spaced over time, each dose comprising an effective amount of pterostilbene).

In some embodiments the pterostilbene is co-administered with one or more non-carbohydrate nutrients. For example, the non-carbohydrate nutrient may be selected from the group consisting of creatine and its salts, esters, amides and chelates; creatinol-O-phosphate; the amino acids leucine, isoleucine, valine, taurine, beta-alanine, arginine, ornithine, aspartic acid, glutamine, glutaric acid, agmatine, citrulline, norvaline, glycine, and cysteine and salts, esters, amides and chelates of said amino acids; ketoisocaproate and sodium, potassium, calcium and magnesium salts thereof; the dipeptides carnitine, anserine and carnosine and salts and esters thereof; and dipeptide-containing proteins.

In some embodiments the pterostilbene is co-administered with one or more carbohydrate nutrients. For example, the carbohydrate nutrient may be selected from the group consisting of: rice oligodextrin; amylose; amylopectin; glucose; maltodextrin; maltose; isomaltulose; leucrose; trehalulose; ribose; trehalose; sucrose; and fructose.

In some embodiments the pterostilbene is co-administered with one or more non-carbohydrate nutrients and one or more carbohydrate nutrients. For example, an effective amount of pterostilbene can be co-administered with both a carbohydrate nutrient and creatine or a salt, ester, amide or chelate thereof.

In some embodiments the pterostilbene is co-administered with one or more compounds not found in a pterostilbene natural source.

In certain embodiments the pterostilbene is co-administered with one or more compounds selected from the group consisting of methylxanthines (e.g., caffeine, theobromine, theophylline), glucuronolactone, animal digestive enzymes (e.g., lipase, bromelain, pancreatin, amylase, lactase), and carbohydrates not found in a pterostilbene natural source (e.g., isomaltulose, trehalose, leucrose, amylose, trehalulose, rice oligodextrin).

The invention further relates to a formulation or composition comprising an amount of pterostilbene effective to increase athletic performance, lower blood sugar level and/or increase lean muscle mass in a human to whom the formulation is administered; and one or more compounds not found in a pterostilbene natural source. In a particular embodiment the formulation or composition comprises creatine or a salt, ester, amide or chelate thereof; in some embodiments the formulation comprises from about 250 mg to about 10,000 mg of creatine or a salt, ester, amide or chelate thereof per dose.

In some embodiments the formulation comprises from about 3 mg to about 25 mg of pterostilbene per dose. In some embodiments the formulation comprises greater than about 0.002 percent pterostilbene by weight, for example from about 0.002 to about 100 percent pterostilbene by weight. In some embodiments the formulation comprises one or more carbohydrate nutrients in addition to pterostilbene. In some embodiments the formulation comprises one or more non-carbohydrate nutrients in addition to pterostilbene.

In some embodiments the formulation comprises, in addition to pterostilbene, one or more compounds selected from the group consisting of methylxanthines (e.g., caffeine, theobromine, theophylline), glucuronolactone, animal digestive enzymes (e.g., lipase, bromelain, pancreatin, amylase, lactase), and carbohydrates not found in pterostilbene natural sources (e.g., isomaltulose, trehalose, leucrose, amylose, trehalulose, rice oligodextrin).

### BRIEF DESCRIPTION OF THE DRAWINGS

FIG. 1 shows pterostilbene (trans-3,5-dimethoxy-4′-hydroxystilbene), molecular formula C₁₆H₁₆O₃, having a molecular weight of about 256.3 g/mol (CAS#537-42-8).

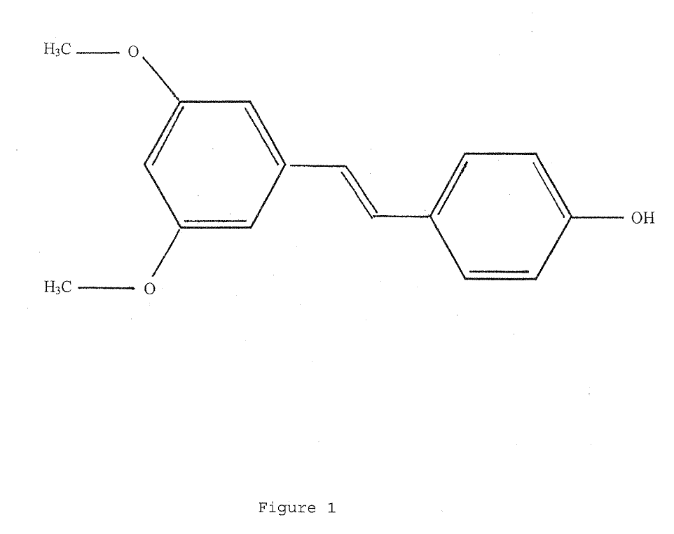

FIG. 2 shows resveratrol (3,4′,5-Trihydroxystilbene), molecular formula C₁₄H₁₂O₃, having a molecular weight of about 228.246 g/mol (CAS# 501-36-0).

### DETAILED DESCRIPTION OF THE INVENTION

Insulin is a polypeptide hormone that has extensive effects on metabolism and other body functions, such as vascular compliance. Insulin causes cells in the liver, muscle, and fat tissue to take up glucose and other nutrients from the blood. Insulin causes glucose to be stored as glycogen in the liver and muscle. Insulin also inhibits the body's use of fat as an energy source. When insulin is absent (or present at low levels), glucose is not taken up by the cells of the body; the body begins to use fat as an energy source (for example, by transfer of lipids from adipose tissue to the liver for mobilization as an energy source). As its level is a central metabolic control mechanism, its status is also used as a control signal to other body systems (such as amino acid uptake by body cells). It has several other anabolic effects throughout the body. When control of insulin levels fails, diabetes mellitus results.

### Action of Insulin

Insulin is produced in the pancreas and released when any of several stimuli is detected. The stimuli include ingested protein and glucose in the blood produced from digested food. Certain carbohydrates produce glucose and thereby increase blood glucose levels. In target cells, carbohydrates initiate a signal transduction, which has the effect of increasing glucose uptake and storage. Ultimately insulin is degraded, terminating the response.

The beta cells in the islets of Langerhans in the pancreas release insulin in two phases. In the first phase insulin release is rapidly triggered in response to increased blood glucose levels. The second phase is a sustained, slow release of newly formed vesicles that are triggered independent of sugar levels. The description of first phase release is as follows:

1. Glucose enters the beta cells through the glucose transporter GLUT2;
2. Glucose goes into the glycolysis and the respiratory cycle where multiple high-energy ATP molecules are produced by oxidation;
3. Dependent on ATP levels, and hence blood glucose levels, the ATP-controlled potassium channels (K+) close and the cell membrane depolarizes;
4. On depolarization, voltage controlled calcium channels (Ca2+) open and calcium flows into the cells'
5. An increased calcium level causes activation of phospholipase C, which cleaves the membrane phospholipid phosphatidyl inositol 4,5-bisphosphate into inositol 1,4,5-triphosphate and diacylglycerol;
6. Inositol 1,4,5-triphosphate (IP3) binds to receptor proteins in the membrane of endoplasmic reticulum (ER). This allows the release of Ca2+ from the ER via IP3 gated channels, and further raises the cell concentration of calcium;
7. Significantly increased amounts of calcium in the cells causes release of previously synthesized insulin, which has been stored in secretory vesicles.

This is the main mechanism for release of insulin. In addition some insulin release takes place generally upon food or other nutrient intake (not limited to glucose or carbohydrate intake), and the beta cells are also somewhat influenced by the autonomic nervous system.

Other substances known to stimulate insulin release include amino acids from ingested proteins, acetylcholine released from vagus nerve endings (parasympathetic nervous system), and glucose-dependent insulinotropic peptide (GIP). Three amino acids (alanine, glycine and arginine) act in a manner similar to glucose by altering the beta cells' membrane potential. Acetylcholine triggers insulin release through phospholipase C, while the last acts through the mechanism of adenylate cyclase.

The sympathetic nervous system (via Alpha2-adrenergic stimulation as demonstrated by the agonists clonidine or methyldopa) inhibits the release of insulin. However, it is worth noting that circulating adrenaline will activate Beta2-Receptors on the beta cells in the pancreatic islets to promote insulin release. This is important, as muscle cannot benefit from the raised blood sugar resulting from adrenergic stimulation (increased gluconeogenesis and glycogenolysis from the low blood insulin: glucagon state) unless insulin is present to allow for GLUT-4 translocation in the tissue. Therefore, beginning with direct innervation, norepinephrine inhibits insulin release via alpha2-receptors; subsequently, circulating adrenaline from the adrenal medulla will stimulate beta2-receptors thereby promoting insulin release.

When the glucose level comes down to the usual physiologic value, insulin release from the beta cells slows or stops. If blood glucose levels drop lower than this, especially to dangerously low levels, release of hyperglycemic hormones (most prominently glucagon from Islet of Langerhans' alpha cells) forces release of glucose into the blood from cellular stores, primarily liver cell stores of glycogen. By increasing blood glucose, the hyperglycemic hormones prevent or correct life-threatening hypoglycemia. Release of insulin is strongly inhibited by the stress hormone norepinephrine (noradrenaline), which leads to increased blood glucose levels during stress. There are special transporter proteins in cell membranes through which glucose and some nutrients from the blood can enter a cell. These transporters are, indirectly, under blood insulin's control in certain body cell types (e.g., muscle cells). Low levels of circulating insulin, or its absence, will prevent glucose and some nutrients from entering those cells (e.g., in Type 1 diabetes). However, more commonly there is a decrease in the sensitivity of cells to insulin (e.g., the reduced insulin sensitivity characteristic of Type 2 diabetes), resulting in decreased glucose and/or nutrient absorption. In either case, there is ‘cell starvation’, weight loss, catabolism, sometimes extreme. In a few cases, there is a defect in the release of insulin from the pancreas. Either way, the effect is, characteristically, the same: elevated blood glucose levels.

Activation of insulin receptors leads to internal cellular mechanisms that directly affect glucose and other nutrient uptake by regulating the number and operation of protein molecules in the cell membrane that transport glucose ad other nutrients into the cell. The genes that specify the proteins that make up the insulin receptor in cell membranes have been identified and the structure of the interior, cell membrane section, and now, finally after more than a decade, the extra-membrane structure of receptor.

It is well known by those skilled in the art that two types of tissues are most strongly influenced by insulin, as far as the stimulation of glucose and other nutrient uptake is concerned: muscle cells (myocytes) and fat cells (adipocytes). The former are important because of their central role in movement, breathing, circulation, etc, and the latter because they accumulate excess food energy against future needs. Together, they account for about two-thirds of all cells in a typical human body.

The actions of insulin on human metabolism include control of cellular intake of certain substances (including nutrients) but most prominently glucose in muscle and adipose tissue (about two thirds of body cells); increase of DNA replication and protein synthesis via control of amino acid uptake; and modification of the activity of numerous enzymes. The actions of insulin at the cellular level include:

1. Increased glycogen synthesis—insulin forces storage of glucose in liver (and muscle) cells in the form of glycogen; lowered levels of insulin cause liver cells to convert glycogen to glucose and excrete it into the blood. This is the clinical action of insulin which is directly useful in reducing high blood glucose levels as in diabetes;

2. Increased fatty acid synthesis—insulin forces fat cells to take in blood lipids which are converted to triglycerides; lack of insulin causes the reverse;

3. Increased esterification of fatty acids—forces adipose tissue to make fats (i.e., triglycerides) from fatty acid esters; lack of insulin causes the reverse;

4. Decreased proteolysis;

5. Decreased lipolysis—forces reduction in conversion of fat cell lipid stores into blood fatty acids; lack of insulin causes the reverse;

6. Decreased gluconeogenesis—decreases production of glucose from non-sugar substrates, primarily in the liver (remember, the vast majority of endogenous insulin arriving at the liver never leaves the liver); lack of insulin causes glucose production from assorted substrates—including amino acids that could be used to increase protein synthesis in skeletal muscle tissue—in the liver and elsewhere;

7. Decreased autophagy—decreased level of degradation and catabolism of damaged organelles. Postprandial levels of insulin inhibit autophagy completely;

8. Increased amino acid and creatine uptake—forces cells to absorb circulating amino acids and creatine; lack of insulin inhibits absorption;

9. Increased potassium uptake—forces cells to absorb serum potassium; lack of insulin inhibits absorption, lowering potassium levels in blood;

10. Arterial muscle tone—forces arterial wall muscle to relax, increasing blood flow, especially in micro arteries; lack of insulin reduces flow by allowing these muscles to contract; and

11. Increase in the secretion of hydrochloric acid by Parietal cells in the stomach.

Insulin currently cannot be taken orally. Like nearly all other proteins introduced into the gastrointestinal tract, it is reduced to fragments (even single amino acid components), whereupon all ‘insulin activity’ is lost. Insulin is usually administered via subcutaneous injection by single-use syringes with needles, an insulin pump, or by repeated-use insulin pens with needles.

Because of the myriad of effects caused directly or indirectly by insulin (some of which are discussed herein), it would be desirable to develop novel oral products that either mimic one or more of the actions of insulin or increase the endogenous production, secretion or activity of insulin. Such products would have utility in treating humans who suffer from conditions resulting from low insulin levels or activity and in increasing the utilization of various nutrients to increase athletic performance.

### Pterostilbene

Pterostilbene is a stilbene found, for example, in deerberry and rabbiteye blueberries, unripe Pinot noir and Botrytis vinifera infected Chardonnay grapes, and immature berries of Pinot and Gamay varieties (J Agric Food Chem 52:4713-9 (2004); J Agric Food Chem 48:6103-6105 (2000); Plant Physiol Biochem 26:603-7 (1988)).

Pterostilbene has been shown to elicit significant anti-oxidant activity in vitro that is comparable to the activity of resveratrol. Research has demonstrated that pterostilbene inhibits cintronellal thermo-oxidation by an EQ value of 335 mM (˜90.9 mg/ml), and that pterostilbene scavenges for 2,2-diphenyL-1-picrylhydrazyl (DPPH) radicals with an EC₅₀ value of about 30 mM (˜7.68 mg/ml) (J Nat Prod 60:609-10 (1997)). Additionally, pterostilbene inhibits 2,2′-azo-bis(2-amidinopropane) (ABAP)-derived peroxyl radicals with a total reactive antioxidant potential of 237+/−58 mM (˜60.7 mg/ml) as compared to resveratrol at 253+/−53 mM (˜57.7 mM/mL) (J Agric Food Chem 50:3453-7 (2002)). Further investigations have demonstrated that pterostilbene protects against lipid peroxidation by reducing thiobarbituric acid reactive substances (TBARS) production by 61% in normal human fibroblasts (J Biol Chem 276:22586-94 (2001)).

Limited research has demonstrated that pterostilbene has cancer chemoprotective properties in both in vitro and in vivo experiments (Neoplasia 1:37-47 (2005); Int J Biochem Cell Biol 37:1709-26 (2005)). Preliminary research has also been undertaken on pterostilbene's ability to inhibit cyclooxygenase (COX) enzymes (J Agric Food Chem 50:3453-7 (2002)).

Pterostilbene and Pterocarpus marsupium extracts that have been shown to contain pterostilbene have reported anti-diabetic properties. Research demonstrated that pterostilbene can lower the blood glucose level in streptozotocin-induced hyperglycemic rats by 42% (J Nat Prod 60:609-10 (1997)). Pterocarpus marsupium, which contains pterostilbene in its heartwood, has also been shown to have anti-hyperglycemic properties and provide significant protection against hypertriglyceridemia and hyperinsulinemia (Diabetes Obes Metab 7:414-20 (2005)).

In the development of new drugs and dietary supplements, the scientific, medical and nutritional communities rely heavily on animal studies that provide a framework for human trials. To assist with this, the U.S. Department of Health & Human Services, Food and Drug Administration, Center for Drug Evaluation and Research (FDA/CDER) published recommendations in July 2005 for estimating the maximum safe starting dose in initial clinical trials for therapeutics in adult healthy volunteers (on the worldwide web at fda.gov/cder/guidance/index.htm). The document, which is incorporated herein in its entirety by reference, outlines a process for deriving the maximum recommended starting dose (MSRD) for first-in-human clinical trials of new molecular entities in adult healthy volunteers and recommends a standardized process by which the MSRD can be selected. Some of the stated purposes of this guiding document are to provide common conversion factors in deriving human equivalent dose (HED) and to delineate a strategy for selecting MSRD for healthy adult volunteers regardless of the projected clinical use.

The recommended process for selecting MSRD requires the determination of “no adverse effects levels” (NOAELs) in the tested animal species; conversion of NOAELs to HED are discussed in this guiding document. The document states that the NOAEL should be identified for each species and then converted to HED using the appropriate scaling factors. For most systemically administered therapeutics, this conversion should be based on the normalization of doses to body surface area. The FDA/CDER guiding document provides tables and algorithmic processes for determining the HED. In particular, page 7 of this document provides a table based on several species of test animals and appropriate formulae to convert these animal doses to HED based on body surface area. When the test animal is a rat, the following formulae are provided:
To convert an animal dose in mg/kg to HED in mg/kg either divide the animal (rat) dose by a factor of 6.2 or multiply the animal dose (rat) by a factor of 0.16—this assumes the HED is for a human with a weight of 60 kg. Alternatively, if the weight the human is not 60 kg, the following formula (Generic Formula) may be employed:

HED=animal dose in mg/kg×(animal weight in kg/human weight in kg) 0.33

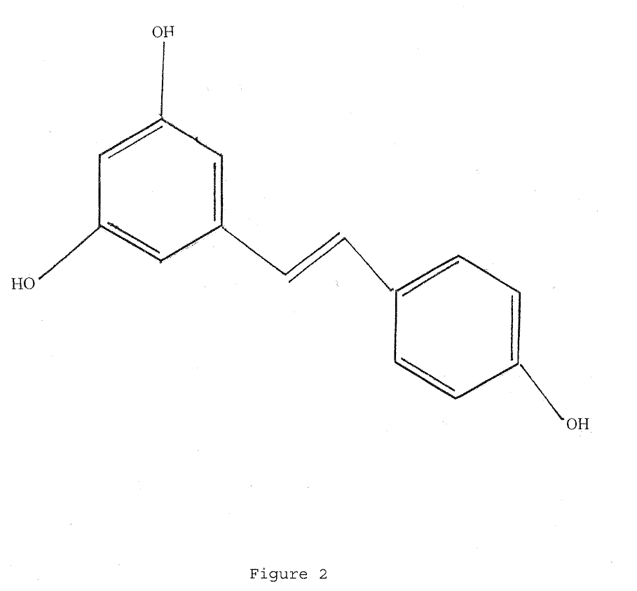

This formula has been successfully employed in determining a HED for resveratrol, another stilbene structurally similar to pterostilbene (FIG. 2).

Thus based on the previous studies administering pterostilbene to rats (having a weight of −250 g) at doses ranging from 10 mg/kg bodyweight up to 40 mg/kg bodyweight with no adverse effects noted in any animal (rat) in any of the aforementioned studies it would be expected that an HED for pterostilbene would be between 1.61 mg/kg of bodyweight (based on pterostilbene dose of 10 mg/kg bodyweight in the rat) and 6.45 mg/kg of bodyweight (based on pterostilbene dose of 40 mg/kg bodyweight in the rat) for a human with a weight of 60 kg. Using the Generic Formula and basing HED determination on a human with a bodyweight of ˜100 kg, the range for pterostilbene would be expected to be between 1.357 mg/kg of bodyweight (based on pterostilbene dose of 10 mg/kg bodyweight in the rat) and 6.542 mg/kg of bodyweight (based on pterostilbene dose of 40 mg/kg bodyweight in the rat). Thus according the FDA/CDER formula, a human with a bodyweight of ˜100 kg would be able to safely use a dose of pterostilbene of about 135 mg to about 654 mg. However, work described herein surprisingly showed this not to be true.

In work described herein using pterostilbene orally in humans (supplied by Chromadex, 10005 Muirlands Boulevard, Suite G, Irvine, Calif. 92618, USA) dosing of oral pterostilbene was initially started at a lower dose than the calculated HED, with the intent of titrating the dose up as needed. The initial oral dose of pterostilbene used in humans in work described herein was 30 mg in a gelatin capsule administered in a single dose to a 35 year old healthy male volunteer who had a bodyweight of approximately 120.53 kg (dose of about 0.248 mg/kg bodyweight).

Within fifteen minutes of oral administration at this dose, the human test subject began to experience symptoms of profound hypoglycemia, including shakiness, dysphoria, and diaphoresis. Testing of the subject's whole blood glucose level by “finger prick” methodology using a commercial glucometer revealed that the test subject's whole blood glucose level had dropped about 50 mg/dL during this time period. The test subject was immediately treated with oral glucose and other carbohydrates and recovered without other incident. Further testing of oral pterostilbene in other human test subjects revealed that pterostilbene is effective in significantly reducing whole blood glucose levels in healthy, adult humans in doses as low as 0.03 mg/kg of bodyweight (actual dose given 3 mg orally) to about 0.165 mg/kg of bodyweight (actual dose given 20 mg orally) without inducing clinically significant side effects including, but not limited to, nausea, dysphoria, diaphoresis. At oral dosing above 0.165 mg/kg (actual dose tested 20 mg) pterostilbene is capable of inducing profound hypoglycemia (e.g., whole blood glucose at or below 55 mg/dL) and the side effects associated with this condition. Accordingly, at the doses predicted by the FDA/CDER formula, oral administration of pterostilbene to humans would likely be fatal.

Additional work described herein addressed the effects of combining oral pterostilbene with various nutrients on athletic performance. Specifically, oral pterostilbene was administered with creatine supplements, and the effect of this combination was compared with the effect of creatine supplements alone. In one test using a 28 year old healthy male volunteer with a body weight of about 102.67 kg, the addition of 5 mg of oral pterostilbene (0.0486 mg/kgbodyweight) to a creatine-containing liquid dietary supplement resulted in approximately an immediate 8% increase in maximal exertion power and approximately an immediate 12% increase in anaerobic work capacity compared with the creatine-containing liquid dietary supplement alone.

Work described herein indicates that 3 mg to 20 mg of pterostilbene added to a creatine-containing supplement improves athletic performance dramatically, with a preferable dose being between about 5 mg and about 10 mg. The addition of exogenous dietary carbohydrates such as glucose, isolmaltulose, leucrose, trehalose, trehalulose, amylose, ribose, and maltodextrin to the creatine-containing supplement allowed for even higher pterostilbene dosing (up to 25 mg of oral pterostilbene) and produced an even more pronounced, immediate increase in athletic performance without the onset of hypoglycemia.

Our experiments revealed that the addition of pterostilbene to other ergogenic, athletic performance-enhancing, non-carbohydrate based, dietary nutrients tends to induce an immediate increase in the athletic performance-enhancing effects of these dietary nutrients when they are orally consumed. While not wishing to be bound by any particular theory, the inventors believe that pterostilbene increases or otherwise effects a change in the rate and quantity of the transfer of these nutrients from the blood plasma into the tissues and organs. Such dietary nutrients include, but are not limited to, creatine and its salts esters, amides and chelates, creatinol-O-phosphate, branched chain amino acids (leucine, isoleucine, valine) and their salts, esters, amides and chelates, branched chain keto-acids including, but not limited to ketoisocaproate and its salts, other ergogenic, performance-enhancing amino acids including, but not limited to, taurine, beta-alanine, arginine, ornithine, aspartic acid, glutamine, glutaric acid, agmatine, citrulline, norvaline, glycine, cysteine and their salts, ester, amides and chelates, performance enhancing dipeptides including, but not limited to, carnitine, anserine and carnosine and their salts and esters and proteins that are known to contain dipeptides such as whey or soy or egg protein isolates or concentrates or any combination of these non-carbohydrate based dietary nutrients.

Further, the addition and oral consumption of one or more exogenous dietary carbohydrates, including but not limited to rice oligodextrin, amylose, amylopectin, glucose, maltodextrin, maltose, isolmaltulose, leucrose, trehalulose, ribose, trehalose, and/or fructose in conjunction with pterostilbene and a non-carbohydrate based dietary nutrient or nutrients simultaneously further increases the athletic performance-enhancing effects of pterostilbene and the non-carbohydrate dietary nutrient(s) by up to 50% and also increases the tolerability of pterostilbene in humans by up to 50%.

The compositions and methods of the present invention may provide significant increase or improvement in athletic performance, e.g., muscle size, and/or muscle strength, and/or muscle endurance in individuals. As used herein, “athletic performance” and/or “athletic functions” refers to the sum of physical attributes which can be dependent to any degree on skeletal muscle contraction. For example, athletic performance and/or athletic functions include, but are not limited to, maximal muscle power, muscular endurance, running speed and endurance, swimming speed and endurance, throwing power, lifting and pulling power. The compositions of the invention can function as insulinogenic agents or insulin mimetics.

While it is expected that the compositions and methods of the present invention will be of particular importance to bodybuilders and other athletes, the usefulness of compositions and methods of the invention is not limited to those groups. Rather, any individual (i.e., human) may beneficially use the compositions and methods of the invention.

The compositions according to the present invention may be employed in methods for supplementing the diet of an individual, e.g., an athlete, and/or for enhancing an individual's muscle mass and/or muscle size and/or strength, and/or endurance. Accordingly, the present invention provides methods of supplementing the dietary intake of an individual comprising administering to the individual an effective amount of a composition (e.g., pterostilbene or a nutritional supplement comprising pterostilbene) according to the present invention to increase athletic performance or athletic function is said individual. The invention also relates to methods of improving athletic performance and/or athletic function in an individual comprising administering an effective amount of a pterostilbene (alone or in combination with other agents, e.g., in a dietary supplements or nutrients) to the individual.

Accordingly, the invention relates in one embodiment to compositions, formulations and methods relating to one or more stilbene-based compounds for use in humans. In particular, compositions and formulations comprising, consisting of or consisting essentially of an effective amount of the stilbene-based insulinogenic compound can improve athletic performance, lower blood glucose levels, and increase lean muscle mass when administered (e.g., orally) to a human. In a particular embodiment the stilbene-based compound is pterostilbene.

Compositions and forumations of the invention can preferably be dietary supplements, dietary formulations, or nutraceuticals. As used herein, the terms “nutrient” and “dietary supplement” and “nutraceutical” are used interchangeably to refer to any substance that is a food or part of a food and provides medical or health benefits, including the prevention and treatment of disease. Hence, compositions falling under the label “nutrient” and “dietary supplement” may range from isolated nutrients, nutritional or dietary supplements, and specific diets, to genetically engineered designer foods, herbal products, and processed foods such as cereals, soups, and beverages. In a more technical sense, the term has been used to refer to a product isolated or purified from foods, and generally sold in medicinal forms not usually associated with foods and demonstrated to have a physiological benefit or provide protection against chronic disease.

Compositions and formulations of the invention will comprise an effective amount of pterostilbene; in some embodiments compositions and formulations of the invention will further comprise at least one (i.e., one or more) compounds or compositions which are not found in a natural source of pterostilbene. In certain embodiments the composition or compound which is not found in a natural source of pterostilbene is an active agent (i.e., is an agent which has a physiological effect either alone or in combination with pterostilbene).

Pterostilbene can be obtained as known in the art by chemical synthesis methods (see, for example, U.S. Pat. No. 7,253,324) or by isolation or extraction from one or more natural sources (see, for example, US Patent Application Publications 20080032372 and 20080124414). In some embodiments the pterostilbene of the invention is administered or exists in a composition or formulation separated from one or more (e.g., from all) compounds with which it exists in a natural source.

As used herein, an “effective amount” in compositions of the present invention is defined as an amount effective, at dosages and for periods of time necessary, to achieve the desired result. For example, the amount of pterostilbene may be effective to increase athletic performance, lower blood sugar levels and/or increase lean muscle mass in a human to whom the formulation is administered. The effective amount of compositions of the invention may vary according to factors such as age, sex, and weight of the individual.

Dosage regime may be adjusted to provide the optimum response. Several divided doses may be administered daily, or the dose may be proportionally reduced as indicated by the exigencies of an individual's situation. As will be readily appreciated, a composition in accordance with the present invention may be administered in a single serving or in multiple servings spaced throughout the day. As will be understood by those skilled in the art, servings need not be limited to daily administration, and may be on an every second or third day or other convenient effective basis. The administration on a given day may be in a single serving or in multiple servings spaced throughout the day depending on the exigencies of the situation. Preferably each dose will comprise an effective amount of pterostilbene.

The invention relates, for example, to a formulation comprising an amount of pterostilbene effective to increase athletic performance in a human to whom the formulation is administered, and creatine or a salt, ester, amide or chelate thereof. The invention also relates to a formulation comprising an amount of pterostilbene effective to reduce blood sugar levels or to increase lean muscle mass in a human to whom it is administered. In some embodiments the effective amount is from about 3 mg to about 25 mg of pterostilbene per dose, more particularly from about 5 mg to about 10 mg of pterostilbene per dose. In other embodiments the formulation comprises from about 250 mg to about 10,000 mg of creatine or a salt, ester, amide or chelate thereof per dose. The formulations and compositions of the invention may, for example, comprise from about 0.002 to about 100 percent pterostilbene by weight. Preferably compositions and formulations of the invention will comprise at least about 0.002 percent pterostilbene by weight.

In some embodiments the formulation comprises one or more carbohydrate nutrients in addition to comprising pterostilbene. For example, the carbohydrate nutrient can be one or more of rice oligodextrin; amylose; amylopectin; glucose; maltodextrin; maltose; isomaltulose; leucrose; trehalulose; ribose; trehalose; sucrose; and fructose.

In other embodiments the formulation comprises one or more non-carbohydrate nutrients in addition to comprising pterostilbene. For example, the non-carbohydrate nutrient may be one or more of creatine and its salts, esters, amides and chelates; creatino1-O-phosphate; the amino acids leucine, isoleucine, valine, taurine, beta-alanine, arginine, ornithine, aspartic acid, glutamine, glutaric acid, agmatine, citrulline, norvaline, glycine, and cysteine and salts, esters, amides and chelates of said amino acids; ketoisocaproate and sodium, potassium, calcium and magnesium salts thereof; the dipeptides carnitine, anserine and carnosine and salts and esters thereof; and dipeptide-containing proteins.

In some embodiments the formulation or compositions of the invention comprise one or more compounds or compositions which are not found in a natural source of pterostilbene, and in a preferred embodiment the one or more compounds or compositions are active agents.

In some embodiments of the invention the composition is formulated as a tablet, capsule, caplet, powder, suspension, gel preparation, aqueous solution, solid food form (e.g., chewable bar or wafer), or liquid dosage form such as elixirs, syrups, dispersed powders, granules or emulsions. In one embodiment the composition is particularly formulated for oral use. Although work described herein focused on oral administration, it is clear that compositions and formulations of the invention may be administered effectively by other routes including, but not limited to, parenteral, buccal, sublingual, rectal, and transdermal. In preferred embodiments the route of administration is oral, and suitable means are especially tablets, caplets, capsules, pulls, suspensions, solutions (e.g., drinks), elixirs that can be produced in ways that are commonly used and familiar to one skilled in the art, with the additives and vehicles that are commonly used. As non-limiting examples, extended release or time release formulations are among technologies known to the skilled artisan and suitable for use with the invention.

In addition, the compositions can be administered or formulated alone or can be formulated with or administered before, concurrent with or after other optional components such as other active ingredients. In some embodiments the composition or formulation comprising pterostilbene contains one or more of the following ingredients, preferably as an active ingredient:
Carbohydrates including, but not limited to, isomaltulose, trehalose, maltodextrin, glucose, sucrose, fructose, lactose, amylose, and/or ribose;
Water soluble vitamins including, but not limited to, Vitamin C, Vitamin B1, Vitamin B2, Vitamin 33, Vitamin B6, Vitamin B12, and/or Vitamin K (and derivatives);
Minerals including, but not limited to, calcium, sodium, potassium, chromium, vanadium, magnesium, and/or iron (and derivatives)(preferably in amounts less than the RDA);
Amino acids including, but not limited to, L-arginine, L-ornithine, L-glutamine, L-tyrosine, L-taurine, L-leucine, L-isoleucine, and/or L-valine (and derivatives);
Nutraceutically acceptable stimulants including, but not limited to, methylxanthines (e.g.—caffeine) and/or glucuronolactone (and derivatives);
Nutraceutically acceptable hypoglycemic agents including, but not limited to, alpha-lipoic acid and its derivatives, cinnamon bark, bitter melon extracts, Gymnema Sylvestre extracts, 4-hydroxy-isoleucine, corosolic acid, and/or D-pinitol (and derivatives);
Creatine and its salts (e.g., creatine monohydrate), esters (e.g., creatine ethyl ester), chelates, amides, ethers (and derivatives);
Adenosine triphosphate and its disodium salt;
Glycerol and glycerol monostearate;
Mannitol;
Sorbitol; and
Dextran.

The compositions may contain pharmaceutically, e.g., nutraceutically, acceptable excipients, according to methods and procedures well known in the art. As used herein, “excipient” refers to substances that are typically of little or no therapeutic value, but are useful in the manufacture and compounding of various pharmaceutical preparations and which generally form the medium of the composition. These substances include, but are not limited to, coloring, flavoring, and diluting agents; emulsifying, dispersing and suspending agents, ointments, bases, pharmaceutical solvents; antioxidants and preservatives; and miscellaneous agents. Suitable excipients are described, for example, in Remington's Pharmaceutical Sciences.

The compositions and formulations according to the present invention can further comprise one or more acceptable carriers. A wide number of acceptable carriers are known in the nutritional supplement arts, and the carrier can be any suitable carrier. The carrier need only be suitable for administration to animals, including humans, and be able to act as a carrier without substantially affecting the desired activity of the composition. Also, the carrier(s) may be selected based upon the desired administration route and dosage form of the composition. For example, the nutritional supplement compositions according to the present invention are suitable for use in a variety of dosage forms, such as liquid form and solid form (e.g., a chewable bar or wafer). In desirable embodiments the compositions and formulations comprise a solid dosage form, such as a tablet or capsule. Examples of suitable carriers for use in tablet and capsule compositions include, but are not limited to, organic and inorganic inert carrier materials such as gelatin, starch, magnesium stearate, talc, gums, silicon dioxide, stearic acid, cellulose, and the like. Desirably, the carrier is substantially inert, but it should be noted that the nutritional supplement compositions of the present invention may contain further active ingredients in addition to pterostilbene.

The invention also relates to a method of increasing athletic performance, lowering blood sugar level and/or increasing lean muscle mass in a human comprising administering an effective amount of a stilbene-based compound to said human. In one embodiment the stilbene-based compound is pterostilbene. In one embodiment the pterostilbene is administered orally.

As used herein, “athletic functions” refers to the sum of physical attributes which can be dependent to any degree on skeletal muscle contraction. For example, athletic functions include, but are not limited to, maximal muscular strength, muscular endurance, running speed and endurance, swimming speed and endurance, throwing power, lifting and pulling power. As used herein, improving or increasing athletic performance or function includes enhancing an individual's muscle mass and/or muscle size and/or strength, and/or endurance.

In certain embodiments the effective amount of pterostilbene is from about 3 mg to about 25 mg per dose, from about 3 mg to about 10 mg per dose, or from about 5 mg to about 10 mg per dose. In certain embodiments the effective amount of pterostilbene is from about 0.03 mg/kg bodyweight of the human to about 0.165 mg/kg bodyweight of the human per dose.

In certain embodiments more than one dose comprising an effective amount of pterostilbene is administered to the human (e.g., two or more doses spaced over time, each dose comprising an effective amount of pterostilbene). The pterostilbene can be co-administered with one or more non-carbohydrate nutrients. For example, the non-carbohydrate nutrient may be selected from the group consisting of creatine and its salts, esters, amides and chelates; creatinol-O-phosphate; the amino acids leucine, isoleucine, valine, taurine, beta-alanine, arginine, ornithine, aspartic acid, glutamine, glutaric acid, agmatine, citrulline, norvaline, glycine, and cysteine and salts, esters, amides and chelates of said amino acids; ketoisocaproate and sodium, potassium, calcium and magnesium salts thereof; the dipeptides carnitine, anserine and carnosine and salts and esters thereof; and dipeptide-containing proteins.

In some embodiments the pterostilbene is co-administered with one or more carbohydrate nutrients. For example, the carbohydrate nutrient may be selected from the group consisting of: rice oligodextrin; amylose; amylopectin; glucose; maltodextrin; maltose; isomaltulose; leucrose; trehalulose; ribose; trehalose; sucrose; and fructose.

In some embodiments the pterostilbene is co-administered with both one or more non-carbohydrate nutrients and one or more carbohydrate nutrients. For example, an effective amount of pterostilbene can be co-administered with both a carbohydrate nutrient and creatine or a salt, ester, amide or chelate thereof.

In some embodiments the pterostilbene is co-administered with one or more compounds not found in a pterostilbene natural source. In some embodiments the pterostilbene is co-administered with one or more compounds selected from the group consisting of methylxanthines (e.g., caffeine, theobromine, theophylline), glucuronolactone, animal digestive enzymes (e.g., lipase, bromelain, pancreatin, amylase, lactase), and carbohydrates not found in a pterostilbene natural source (e.g., isomaltulose, trehalose, leucrose, amylose, trehalulose, rice oligodextrin).

As used herein, “co-administration” includes, but is not limited to, concurrent administration of multiple compositions, either as separate compositions or in admixture. Co-administration also includes administration of the multiple compositions proximal in time, as long as the co-administered compositions exhibit the synergistic or enhanced functional effect observed when the compositions are administered concurrently. The synergistic effect or enhanced effect need not be of the same scope or magnitude as observed upon concurrent administration.

In particular, co-administration of an effective amount of pterostilbene and a non-carbohydrate nutrient exhibits an improved effect on athletic performance as compared with either nutrient alone. Moreover this co-administration exhibits an enhanced or synergistic effect on athletic performance which is greater than the additive effect of the nutrients. Addition of a carbohydrate nutrient further enhances this effect. Particularly, and without limitation, increased muscle volumization occurs, ADP to ATP regeneration increases, and/or glycogen synthesis in muscle increase.

The embodiments set forth in the present application are provided only to illustrate various aspects of the invention, and additional embodiments and advantages of the food supplements and methods of the present invention will be apparent to those skilled in the art. The articles “a” and “an” as used herein, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should be understood that any embodiment or aspect of disclosed invention may be freely combined with one or more other embodiments of the invention unless otherwise indicated; exhaustive combinations of disclosed embodiments have not in every instance been specifically set forth verbatim but are intended to be encompassed by this disclosure. It should also be understood that any embodiment or aspect of the invention can be explicitly excluded from the claims, regardless of whether the specific exclusion is recited in the specification. The teachings of all references cited herein are incorporated herein by reference in their entirety.

### EXAMPLES

Non-limiting examples of pterostilbene used with non-carbohydrate dietary nutrients and pterostilbene used with non-carbohydrate dietary nutrients plus exogenous carbohydrates are provided in the examples below. The servings set forth in these examples are designed for an athlete with a body mass of about 70 kilograms. Daily values can be increased or decreased, depending on the body mass of the individual athlete and individual needs and requirements.

### Example 1

In this example, an athlete consumes one serving of the food supplement described herein daily; typically the athlete will consume a serving of the food supplement about 30-60 minutes before exercise. Each serving is about 85.855 grams and contains the following:

Pterostilbene 0.005 grams; Creatine Monohydrate 3.00 grams; Magnesium Creatine Chelate 2.00 grams; L-Leucine 5.00 grams; Propionyl-L-Carnitine 1.00 gram; Ubiquinone 0.100 grams; L-Taurine 3.00 grams; L-Glutamine 7.50 grams; L-Tyrosine 2.00 grams; Disodium ATP 0.200 grams; Partially Hydrolyzed Guar Gum 5.00 grams; Isomaltulose 15.00 grams; Trehalose 15.00 grams; Glucose 15.00 grams; Calcium Phosphate 3.00 grams; Calcium Citrate 2.00 grams; Calcium Bicarbonate 5.00 grams; Carnosine 2.00 grams and Potassium R-a-Lipoic Acid 0.050 grams.
Each 85.855 gram serving is administered as a powder dissolved into about 500-750 milliliters of water or fruit juice to provide a liquid drink.

### Example 2

In this example, an athlete consumes two servings of the food supplement as described herein daily; typically the athlete will consume one serving of the food supplement about 30-60 minutes before exercise or athletic activity and the second serving of the food supplement immediately after the cessation of exercise or athletic activity. Each serving is about 99.005 grams and contains the following:

Pterostilbene 0.010 grams; Creatine Monohydrate 3.00 grams; Maltodextrin 50.00 grams; and Whey Protein Concentrate 46.00 grams
Each approximately 99.010 gram serving is mixed in about 500-1000 milliliters of water or fruit juice to provide a liquid drink.

### Example 3

In this example, an athlete consumes three servings of the food supplement as described herein daily; typically the athlete will consume one serving of the food supplement about 30-60 minutes before exercise or athletic activity, the second serving of the food supplement immediately after the cessation of exercise or athletic activity, and an additional serving 6-8 hours later. Each serving is about 19.8021 grams and contains the following:

Pterostilbene 0.0021 grams (2.100 milligrams); Leucine 3.00 grams; Isoleucine 1.500 grams; Valine 1.50 grams, b-alanine 0.800 grams, Taurine 5.00 grams, Glutamine 5.00 grams.
Each approximately 19.8021 gram serving is mixed in about 300-500 milliliters of water or fruit juice to provide a liquid drink.

What is claimed is:

1. A method of increasing athletic performance in a human comprising administering an effective amount of pterostilbene to said human, wherein said effective amount is less than or equal to about 25 mg per dose.

2. A method of lowering the blood sugar level in a human comprising administering an effective amount of pterostilbene to said human, wherein said effective amount is less than or equal to about 25 mg per dose.

3. A method of increasing lean muscle mass in a human comprising administering an effective amount of pterostilbene to said human, wherein said effective amount is less than or equal to about 25 mg per dose.

4. A method according to claim 1, 2 or 3 wherein the pterostilbene is administered orally.

5. A method according to claim 1, 2 or 3 wherein the effective amount of pterostilbene is from about 3 mg to about 25 mg per dose.

6. A method according to claim 1, 2 or 3 wherein the effective amount of pterostilbene is from about 5 mg to about 10 mg per dose.

7. A method according to claim 1, 2 or 3 wherein the effective amount of pterostilbene is from about 0.03 mg/kg bodyweight of the human to about 0.165 mg/kg bodyweight of the human per dose.

8. A method according to claim 1, 2 or 3 wherein more than one dose comprising an effective amount of pterostilbene is administered to the human.

9. A method according to claim 1, 2 or 3 wherein the pterostilbene is co-administered with one or more non-carbohydrate nutrients.

10. A method according to claim 9 wherein the non-carbohydrate nutrient is selected from the group consisting of: creatine and its salts, esters, amides and chelates; creatinol-O-phosphate; the amino acids leucine, isoleucine, valine, taurine, beta-alanine, arginine, ornithine, aspartic acid, glutamine, glutaric acid, agmatine, citrulline, norvaline, glycine, and cysteine and salts, esters, amides and chelates of said amino acids; ketoisocaproate and sodium, potassium, calcium and magnesium salts thereof; the dipeptides carnitine, anserine and carnosine and salts and esters thereof; and dipeptide-containing proteins.

11. A method according to claim 9 wherein the non-carbohydrate nutrient is selected from the group consisting of creatine and its salts, esters, amides and chelates.

12. A method according to claim 1, 2 or 3 wherein the pterostilbene is co-administered with one or more carbohydrate nutrients.

13. A method according to claim 12 wherein the carbohydrate nutrient is selected from the group consisting of: rice oligodextrin; amylose; amylopectin; glucose; maltodextrin; maltose; isomaltulose; leucrose; trehalulose; ribose; trehalose; sucrose; and fructose.

14. A method according to claim 1, 2 or 3 wherein the pterostilbene is co-administered with one or more non-carbohydrate nutrients and one or more carbohydrate nutrients.

15. A method according to claim 14 wherein the non-carbohydrate nutrient is selected from the group consisting of: creatine and its salts, esters, amides and chelates; creatinol-O-phosphate; the amino acids leucine, isoleucine, valine, taurine, beta-alanine, arginine, ornithine, aspartic acid, glutamine, glutaric acid, agmatine, citrulline, norvaline, glycine, and cysteine and salts, esters, amides and chelates of said amino acids; ketoisocaproate and sodium, potassium, calcium and magnesium salts thereof; the dipeptides carnitine, anserine and carnosine and salts and esters thereof; and dipeptide-containing proteins.

16. A method according to claim 14 wherein the non-carbohydrate nutrient is selected from the group consisting of creatine and its salts, esters, amides and chelates.

17. A method according to claim 14 wherein the carbohydrate nutrient is selected from the group consisting of: rice oligodextrin; amylose; amylopectin; glucose; maltodextrin; maltose; isolmaltulose; leucrose; trehalulose; ribose; trehalose; sucrose; and fructose.

18. A method according to claim 1, 2 or 3 wherein the pterostilbene is co-administered with a carbohydrate nutrient and with creatine or a salt, ester, amide or chelate thereof.

19. A method according to claim 1, 2 or 3 wherein the pterostilbene is co-administered with one or more compounds not found in a natural source of pterostilbene.

20. A formulation comprising:
an amount of pterostilbene effective to increase athletic performance in a human to whom the formulation is administered; and
one or more compounds not found in a natural source of pterostilbene.

21. A formulation according to claim 20 wherein the formulation comprises from about 3 mg to about 25 mg of pterostilbene per dose.

22. A formulation according to claim 20 wherein the formulation comprises from about 250 mg to about 10,000 mg of creatine or a salt, ester, amide or chelate thereof per dose.

23. A formulation according to claim 20 wherein the formulation comprises at least about 0.002 percent pterostilbene by weight.

24. A formulation according to claim 20 comprising one or more carbohydrate nutrients in addition to pterostilbene.

25. A formulation according to claim 20 comprising one or more non-carbohydrate nutrients in addition to pterostilbene.

26. A formulation according to claim 20 comprising one or more compounds selected from the group consisting of methylxanthines, glucuronolactone, animal digestive enzymes, and carbohydrates not found in a pterostilbene natural source.

27. A method according to claim 1, 2 or 3 wherein the pterostilbene is co-administered with one or more compounds selected from the group consisting of methylxanthines, glucuronolactone, animal digestive enzymes, and carbohydrates not found in a pterostilbene natural source.
